(19) United States
(12) Patent Application Publication
(10) Pub. No.: US 2010/0035868 A1
(43) Pub. Date: Feb. 11, 2010

(54) METHODS OF TREATMENT OF UTERINE PATHOLOGICAL CONDITIONS

(75) Inventor: Henry Nicolas Jabbour, Edinburgh (GB)

Correspondence Address: NIXON PEABODY LLP - PATENT GROUP, 1100 CLINTON SQUARE, ROCHESTER, NY, 14604, US

(73) Assignee: MEDICAL RESEARCH COUNCIL, London (GB)

(21) Appl. No.: 12/574,451

(22) Filed: Oct. 6, 2009

Related U.S. Application Data
(63) Continuation of application No. 10/267,129, filed on Oct. 8, 2002.
(60) Provisional application No. 60/333,562, filed on Nov. 27, 2001.

(30) Foreign Application Priority Data
Oct. 8, 2001 (GB) 0124124.9

(51) Int. Cl. A61K 31/5415; A61K 31/18

(52) U.S. Cl. 514/226.5; 514/605

(57) ABSTRACT

A method of treating or preventing a pathological condition of the uterus in an individual the method comprising administering to the individual any one or more of an inhibitor of cyclooxygenase-2 (COX-2), an inhibitor of prostaglandin E synthase (PGES), or an EP2 or EP4 receptor antagonist. Typically, the pathological condition is uterine cancer, fibroids or endometriosis.

This application is a continuation of U.S. patent application Ser. No. 10/267,129, filed Oct. 8, 2002, which claims the benefit of U.S. Provisional Patent Application Ser. No. 60/333,562, filed Nov. 27, 2001, and Great Britain Patent Application No. 01/24124.9 filed Oct. 8, 2001, all of which are hereby incorporated by reference in their entirety.

The present invention relates to methods of treatment, and in particular methods of treating uterine pathological conditions.

Pathological conditions of the uterus represent a serious health problem in women, particularly women of the Western world. Such pathological conditions include uterine carcinoma, and endometrial or myometrial pathological conditions such as endometriosis (endometrial) and fibroids (myometrial).

Cyclooxygenase (COX) enzymes, also called prostaglandin endoperoxide synthase, (PGHS), catalyse the rate limiting step in the conversion of arachidonic acid to prostaglandin H₂ (PGH₂). In turn PGH₂ serves as a substrate for specific prostaglandin synthase enzymes that synthesise the natural prostaglandins. These are named according to the prostaglandin they produce such that prostaglandin D₂ is synthesised by prostaglandin-D-synthase, prostaglandin E₂ (PGE₂) by prostaglandin-E-synthase (PGES) and prostaglandin F_{2I} by prostaglandin-F-synthase. To-date, there are two identified isoforms of the COX enzyme, COX-1 and COX-2 (DeWitt, 1991). COX-1 is constitutively expressed in many tissues and cell types and generates prostaglandins for normal physiological function (Herschman, 1996). By contrast, the expression of COX-2 is rapidly induced following stimulation of quiescent cells by growth factors, oncogenes, carcinogens and tumour-promoting phorbol esters (Herschman, 1996; Subbaramaiah et al., 1996). In addition, two isoforms of PGES have been isolated; a microsomal glutathione-dependent inducible PGES (mPGES) and a constitutive cytosolic glutathione dependent PGES (Jakobsson et al., 1999; Tanioka et al., 2000). In vitro studies support the idea that COX-2 and possibly PGE₂ are involved in neoplastic transformation of certain epithelial cells and subsequently carcinogenesis. Over-expression of COX-2 and PGE₂ synthesis in rat intestinal epithelial cells increases their proliferation rate, resistance to apoptosis, and their invasiveness by suppressing the transcription of target genes that may be involved in cellular growth/transformation and adhesion (Tsujii & DuBois, 1995). In addition, it has been proposed recently that COX-2 and PGE₂ promote cancer development and invasiveness by mediating the transcription of angiogenic factors that induce both migration of endothelial cells and their arrangement into tubular structures (Tsujii et al., 1998; Jones et al., 1999b).

Cyclooxygenases have been studied in various cancers, but the picture that has emerged to date is confusing and it is not possible to predict the role of COX-1 or COX-2 in any particular cancer. For example, both COX-1 and COX-2 have been shown to be highly expressed in lung cancer in the mouse (Bauer et al (2000) Carcinogenesis 21, 543-550) whereas Rioux & Castonguay (2000) Carcinogenesis 21, 1745-1751 indicates that COX-1 is induced by tobacco carcinogens in human macrophages and is correlated with NFκB activation. According to Doré et al (1998) J. Histochem. Cytochem. 46, 77-84 COX-1 but not COX-2 is expressed in human ovarian adenocarcinomas. According to Ryu et al (2000) Gynecologic Oncology 76, 320-325 COX-2 expression is high in stage IB cervical cancer whereas COX-1 was expressed without regard to location of the tumour cells or type of cancer cell and the authors indicate that COX-1 is unrelated to apoptosis, tumourigenesis and tumour invasion mechanisms. Kulkarni et al (2001) Clin. Cancer Res. 7, 429-434 indicates that COX-2 is overexpressed in human cervical cancer.

PGE₂ mediates its effect on target cells through interaction with different isoforms of seven transmembrane G protein coupled receptors (GPCR) which belong to the rhodopsin family of serpentine receptors. Four main PGE₂ receptor subtypes have been identified (EP1, EP2, EP3 and EP4) which utilise alternate and in some cases opposing intracellular signalling pathways (Coleman et al., 1994). This diversity of receptors with opposing action may confer a homeostatic control on the action of PGE₂ that is released in high concentrations close to its site of synthesis (Ashby, 1998). To-date, the role of the different PGE₂ receptors, their divergent intracellular signalling pathways, as well as their respective target genes involved in mediating the effects of PGE₂ on normal or neoplastically transformed endometrial epithelial cells remain to be elucidated.

Epithelial cells of the human endometrium are highly vulnerable to neoplastic transformation. In the western world, endometrial carcinoma is the most common gynaecologic malignancy. Endometrial cancer can arise from several cell types but the glandular epithelium is the most common progenitor (adenocarcinomas account for 80-90% of uterine tumours). Endometrial cancer is predominantly a post-menopausal disease where incidence is uncommon below the age of forty and peaks by about seventy years of age. The incidence of endometrial cancer has been increasing steadily in the Western world during the last fifty years and this has been attributed largely to increased life expectancy and improved detection methods (Gordon & Ireland, 1994; Mant & Vessey, 1994).

Surprisingly we found in the study described in Example 1 that COX-2 and mPGES expression and PGE₂ synthesis are up-regulated in adenocarcinoma of the human uterus. Expression of these factors was localised to the neoplastic epithelial cells of the uterine carcinoma tissues as well as the endothelial cells of the microvasculature. This is associated with an overexpression, and signalling of the EP2 and EP4 receptors in the carcinoma tissue.

A first aspect of the invention provides a method of treating a pathological condition of the uterus in an individual the method comprising administering to the individual any one or more of an inhibitor of cyclooxygenase-2 (COX-2), an inhibitor of prostaglandin E synthase (PGES), or an EP2 or EP4 receptor antagonist.

The pathological condition of the uterus may be any pathological condition wherein expression of COX-2, and synthesis of PGE2 is found, and wherein expression of EP2 and EP4 receptors is found. Typically the pathological condition of the uterus is any one of uterine carcinoma, an endometrial pathological condition such as endometriosis including adenomyosis, or a myometrial pathological condition such as fibroids (leiomyomas) or leiomyosarcomas which are fibroids which have become malignant. Thus, typically, the uterine pathological condition is one which is associated with abnormal growth of cells of the myometrium or endometrium. Endometriosis is the ectopic implantation and growth of endometrium and can therefore be considered as abnormal growth cells of the endometrium as defined. Adenomyosis is a form of endometriosis where the ectopic endometrium is implanted in the myometrium.

It is particularly preferred if the method of the invention is used to treat endometrial carcinoma.

In one embodiment of the invention, the individual is administered a COX-2 inhibitor. Preferably the inhibitor is selective for COX-2.

The compound may selectively inhibit COX-2 function at any level. Suitably, the compound selectively inhibits COX-2 enzyme activity.

By “selectively inhibits COX-2 enzyme activity” we mean that the compound preferably inhibits COX-2 in preference to other cyclo-oxygenase enzymes, in particular in preference to cyclo-oxygenase-1 (COX-1). The COX-1 gene and the sequence of its polypeptide product are described in Yokoyama and Tanabe (1989) Biochem. Biophys. Res. Comm. 165, 888-894 incorporated herein by reference. COX-1 is also called PGHS-1. The COX-2 gene and the sequence of its polypeptide product are described in O'Banion et al (1991) J Biol. Chem. 266, 23261-23267 incorporated herein by reference. COX-2 is also called PGHS-2.

Conveniently, the compound which selectively inhibits COX-2 enzyme activity is at least ten times better at inhibiting COX-2 than COX-1; preferably it is at least fifty times better; preferably it is at least one hundred times better; still more preferably it is at least one thousand times better and in greater preference it is at least ten thousand times better.

It is most preferred if the compound has substantially no inhibitory activity against the COX-1 enzyme.

Conveniently, the compound selectively inhibits COX-2 enzyme production. The compound may, for example, selectively prevent transcription of the COX-2 or it may selectively prevent translation of the COX-2 message.

By “selectively inhibits COX-2 enzyme production” we mean that the compound preferably inhibits the production of COX-2 in preference to other cyclo-oxygenases, in particular in preference to the production of COX-1.

Conveniently, the compound which selectively inhibits COX-2 enzyme production is at least ten times better at inhibiting COX-2 production than COX-1 production; preferably it is at least fifty times better; more preferably it is at least one hundred times better; more preferably still it is at least one thousand times better; and in greater preference it is at least ten thousand times better.

It is most preferred if the compound has substantially no inhibitory activity against COX-1 enzyme production.

A particularly preferred embodiment is wherein the compound is any one of nimesulide, 4-hydroxynimesulide, flosulide, and meloxicam.

Nimesulide is N-(4-nitro-2-phenoxyphenyl)methane sulfonamide (also called 4-nitro-2-phenoxymethanesulfonanilide). Nimesulide is 100-fold more specific for COX-2 inhibition than for COX-1 inhibition. Nimesulide is manufactured by Boehringer.

Flosulide is 6-(2,4-difluorophenoxy)-5-methyl sulphonylamino-1-indanone (also known as N-6-(2,4-difluorophenoxy)-1-oxo-indan-5-yl methane-sulphonamide). Flosulide is 1000-fold more specific for COX-2 inhibition than for COX-1 inhibition. Flosulide is manufactured by Ciba Geigy.

Meloxicam is 4-hydroxy-2-methyl-N-(5-methyl-2-thiazolyl)-2H-1,2-benzothiazine-3-carboxamide 1,1-dioxide. Meloxicam is 1000-fold more specific for COX-2 inhibition than for COX-1 inhibition. Meloxicam is manufactured by Boehringer.

The synthesis of nimesulide is well known and is described in U.S. Pat. No. 3,840,597; the synthesis of flosulide is well known and is described in GB 2 092 144; and the synthesis of meloxicam is well known and is described in U.S. Pat. No. 4,233,299.

Other COX-2-specific inhibitors which may be useful in the practice of the invention include:

L 475 L337 which is 500-fold more specific for COX-2 inhibition than for COX-1 inhibition. This is manufactured by Merck Frost.

Vioxx, sold by Merck, is also a suitable COX-2 inhibitor.

SC 58125 Celecoxib which is 100-fold more specific for COX-2 inhibition than for COX-1 inhibition. Celecoxib is manufactured by Searle.

NS 398 which is manufactured by Taisho and which is very highly selective for COX-2.

DuP 697, which is COX-2-selective and is manufactured by DuPont.

Nimesulide, flosulide and meloxicam are COX-2 enzyme inhibitors, probably competitive inhibitors.

When the patient is administered a COX-2 inhibitor it is preferred if the patient is not also administered the aromatase inhibitor exemestane.

In a further embodiment of the invention, the individual is administered an inhibitor of PGES. It has been reported by Thorén & Jakobsson (2000) Eur. J Biochem. 267, 6428-6434 (incorporated herein by reference) that NS-398, sulindac sulphide and leukotriene C₄ inhibit PGES activity with IC₅₀ values of 20TM, 80TM and 5TM, respectively.

In a still further embodiment of the invention, the individual is administered an antagonist of an EP2 receptor or an antagonist of an EP4 receptor.

The prostaglandin EP2 receptor antagonist may be any suitable EP2 receptor antagonist. Similarly, the prostaglandin EP4 receptor antagonist may be any suitable EP4 receptor antagonist. By “suitable” we mean that the antagonist is one which may be administered to the patient. The receptor antagonists are molecules which bind to their respective receptors, compete with the natural ligand (PGE₂) and inhibit the initiation of the specific receptor-mediated signal transduction pathways. The receptor antagonists are typically selective to the particular receptor and typically have a higher binding affinity to the receptor than the natural ligand. Although antagonists with a higher affinity for the receptor than the natural ligand are preferred, antagonists with a lower affinity may also be used, but it may be necessary to use these at higher concentrations. Preferably, the antagonists bind reversibly to their cognate receptor. Typically, antagonists are selective for a particular receptor and do not affect the other receptor; thus, typically, an EP2 receptor antagonist binds the EP2 receptor but does not substantially bind the EP4 receptor, whereas an EP4 receptor antagonist binds the EP4 receptor but does not substantially bind the EP2 receptor. Preferably, the EP2 or EP4 receptor antagonist is selective for the particular receptor subtype. By this is meant that the antagonist has a binding affinity for the particular receptor subtype which is at least ten-fold higher than for at least one of the other EP receptor subtypes. Thus, selective EP4 receptor antagonists have at least a ten-fold higher affinity for the EP4 receptor than any of the EP1, EP2 or EP3 receptor subtypes.

It is particularly preferred that the EP2 or EP4 receptor antagonist is selective for its cognate receptor.

The EP2 or EP4 receptor antagonists are typically administered in an effective amount to combat the pathological condition of the uterus. Thus, the antagonists may be used to alleviate symptoms (ie are used palliatively) or may be used to treat the condition. The antagonist may be administered prophylactically (and by “treating” we include prophylactic treatment). The antagonist may be administered by any suitable route, and in any suitable form. It is desirable to administer an amount of the EP2 or EP4 receptor antagonist that is effective in preventing or alleviating or ameliorating or curing the pathological condition of the uterus.

EP2 receptor antagonists include AH6809 (Pelletier et al (2001) Br. J. Pharmacol. 132, 999-1008).

EP4 receptor antagonists include AH23848B (developed by Glaxo) and AH22921X (Pelletier et al (2001) Br. J. Pharmacol. 132, 999-1008. The chemical name for AH23848B is ([1alpha(z), 2beta5alpha]-(±)-7-[5-[[(1,1′-biphenyl)-4-yl]methoxy]-2-(4-morpholinyl)-3-oxo-cyclopentyl]-4-heptenoic acid) (see Hillock & Crankshaw (1999) Eur. J. Pharmacol. 28, 99-108). EP4RA (Li (2000) Endocrinology 141, 2054-61) is an EP(4)-selective ligand (Machwate et al (2001) Mol. Pharmacol. 60: 36-41). The omega-substituted prostaglandin E derivatives described in WO 00/15608 (EP 1 114 816) (Ono Pharm Co Ltd) bind EP4 receptors selectively and may be EP4 receptor antagonists.

Peptides described in WO 01/42281 (Hopital Sainte-Justine) eg: IFTSYLECL (SEQ ID NO: 1), IFASYECL (SEQ ID NO: 2), IFTSAECL (SEQ ID NO: 3), IFTSYEAL (SEQ ID NO: 4), ILASYECL (SEQ ID NO: 5), IFTSTDCL (SEQ ID NO: 6), XTSYEAL (where X is 4-biphenyl alanine) (SEQ ID NO: 7), XTSYEAL (where X is homophenyl alanine) (SEQ ID NO: 8) are also described as EP4 receptor antagonists, as are some of the compounds described in WO 00/18744 (Fujisawa Pharm Co Ltd). The 5-thia-prostaglandin E derivatives described in WO 00/03980 (EP 1 097 922) (Ono Pharm Co Ltd) may be EP4 receptor antagonists.

EP4 receptor antagonists are also described in WO 01/10426 (Glaxo), WO 00/21532 (Merck) and GB 2 330 307 (Glaxo).

WO 00/21532 describes the following as EP4 receptor antagonists:

5-butyl-2,4-dihydro-4-[[2′-[N-(3-chloro-2-thiophenecarbonyl)sulfamoyl]biphenyl-4-yl]methyl]-2-{2-(trifluoromethyl)phenyl]-1,2,4-triazol-3-one potassium salt;

5-butyl-2,4-dihydro-4-[[2′-[N-(2-methyl-3-furoyl)sulfamoyl]biphenyl-4-yl]methyl]-2-{2-(trifluoromethyl)phenyl]-1,2,4-triazol-3-one;

5 -butyl-2,4-dihydro-4-[[2′-[N-(3-methyl-2-thiophenecarbonyl)sulfamoyl]biphenyl-4-yl]methyl]-2-{2-(trifluoromethyl)phenyl]-1,2,4-triazol-3-one;

5-butyl-2,4-dihydro-4-[[2′-[N-(2-thiophenecarbonyl)sulfamoyl]biphenyl-4-yl]methyl]-2-{2-(trifluoromethyl)phenyl]-1,2,4-triazol-3-one;

5-butyl-2,4-dihydro-4-[[2′-[N-[2-(methypyrrole)carbonyl]sulfamoyl]biphenyl-4-yl]methyl]-2-{2-(trifluoromethyl)phenyl]-1,2,4-triazol-3-one.

GB 2 330 307 describes [1α(Z), 2β,5α]-(±)-7-[5-[[(1,1′-biphenyl)-4-yl]methoxy]-2-(4-morpholinyl)-3-oxocyclopentyl]-4-heptenoic acid and [1R[1α(z),2β,5α]]-(−)-7-[5-[[(1,1′-biphenyl)-4-yl]methoxy]-2-(4-morpholinyl)-3-oxocyclopentyl]-4-heptenoic acid.

WO 00/18405 (Pharmagene) describes the EP4 receptor antagonists AH22921 and AH23848 (which are also described in GB 2 028 805 and U.S. Pat. No. 4,342,756). WO 01/72302 (Pharmagene) describes further EP4 receptor antagonists, for example those described by reference to, and included in the general formula (I) shown on page 8 et seq.

All of these references to EP2 and EP4 receptor antagonists are incorporated herein by reference.

It will be appreciated that one or more EP2 receptor antagonists, or one or more EP4 receptor antagonists, may be administered to the patient. It will also be appreciated that a combination of one or more EP2 or EP4 receptor antagonists may be administered to the patient.

It may be advantageous to administer to the individual a combination of one or more of the COX-2 inhibitor or the PGES inhibitor or antagonist of EP2 receptor or antagonist of EP4 receptor. These may all be considered “treatment agents” of the invention.

The treatment agents are administered in an effective amount to combat the undesired pathological condition of the uterus. Thus, the treatment agents may be used to alleviate symptoms (ie are used palliatively) or may be used to treat the condition or may be used prophylactically to prevent the condition. The treatment agent may be administered by any suitable route, and in any suitable form. The aforementioned treatment agents for use in the invention or a formulation thereof may be administered by any conventional method including oral and parenteral (eg subcutaneous or intramuscular) injection. The treatment may consist of a single dose or a plurality of doses over a period of time. The dose to be administered is determined upon consideration of age, body weight, mode of administration, duration of the treatment and pharmacokinetic and toxicological properties of the treatment agent or agents. The treatment agents are administered at a dose (or in multiple doses) which produces a beneficial therapeutic effect in the patient. Typically, the treatment agents are administered at a dose the same as or similar to that used when the treatment agent is used for another medical indication. In any event, the dose suitable for treatment of a patient may be determined by the physician.

Whilst it is possible for a treatment agent of the invention to be administered alone or in combination with other said treatment agents, it is preferable to present it or them as a pharmaceutical formulation, together with one or more acceptable carriers. The carrier(s) must be “acceptable” in the sense of being compatible with the treatment agent of the invention and not deleterious to the recipients thereof. Typically, the carriers will be water or saline which will be sterile and pyrogen free.

The formulations may conveniently be presented in unit dosage form and may be prepared by any of the methods well known in the art of pharmacy. Such methods include the step of bringing into association the treatment agent or agents with the carrier which constitutes one or more accessory ingredients. In general the formulations are prepared by uniformly and intimately bringing into association the active ingredient (ie treatment agent or agents) with liquid carriers or finely divided solid carriers or both, and then, if necessary, shaping the product.

Formulations in accordance with the present invention suitable for oral administration may be presented as discrete units such as capsules, cachets or tablets, each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

A tablet may be made by compression or moulding, optionally with one or more accessory ingredients. Compressed tablets may be prepared by compressing in a suitable machine the active ingredient in a free-flowing form such as a powder or granules, optionally mixed with a binder (eg povidone, gelatin, hydroxypropylmethyl cellulose), lubricant, inert diluent, preservative, disintegrant (eg sodium starch glycolate, cross-linked povidone, cross-linked sodium carboxymethyl cellulose), surface-active or dispersing agent. Moulded tablets may be made by moulding in a suitable machine a mixture of the powdered compound moistened with an inert liquid diluent. The tablets may optionally be coated or scored and may be formulated so as to provide slow or controlled release of the active ingredient therein using, for example, hydroxypropylmethylcellulose in varying proportions to provide desired release profile.

Formulations suitable for topical administration in the mouth include lozenges comprising the active ingredient in a flavoured basis, usually sucrose and acacia or tragacanth; pastilles comprising the active ingredient in an inert basis such as gelatin and glycerin, or sucrose and acacia; and mouth-washes comprising the active ingredient in a suitable liquid carrier. Buccal administration is also preferred.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules and tablets of the kind previously described.

Preferred unit dosage formulations are those containing a daily dose or unit, daily sub-dose or an appropriate fraction thereof, of an active ingredient.

It should be understood that in addition to the ingredients particularly mentioned above the formulations of this invention may include other agents conventional in the art having regard to the type of formulation in question, for example those suitable for oral administration may include flavouring agents.

Certain EP2 and EP4 receptor antagonists are proteins or peptides. Proteins and peptides may be delivered using an injectable sustained-release drug delivery system. These are designed specifically to reduce the frequency of injections. An example of such a system is Nutropin Depot which encapsulates recombinant human growth hormone (rhGH) in biodegradable microspheres that, once injected, release rhGH slowly over a sustained period.

The protein and peptide can be administered by a surgically implanted device that releases the drug directly to the required site. For example, Vitrasert releases ganciclovir directly into the eye to treat CMV retinitis. The direct application of this toxic agent to the site of disease achieves effective therapy without the drug's significant systemic side-effects.

Electroporation therapy (EPT) systems can also be employed for the administration of proteins and peptides. A device which delivers a pulsed electric field to cells increases the permeability of the cell membranes to the drug, resulting in a significant enhancement of intracellular drug delivery.

Proteins and peptides can be delivered by electroincorporation (EI). EI occurs when small particles of up to 30 microns in diameter on the surface of the skin experience electrical pulses identical or similar to those used in electroporation. In EI, these particles are driven through the stratum corneum and into deeper layers of the skin. The particles can be loaded or coated with drugs or genes or can simply act as “bullets” that generate pores in the skin through which the drugs can enter.

An alternative method of protein and peptide delivery is the ReGel injectable system that is thermo-sensitive. Below body temperature, ReGel is an injectable liquid while at body temperature it immediately forms a gel reservoir that slowly erodes and dissolves into known, safe, biodegradable polymers. The EP2 or EP4 receptor antagonist is delivered over time as the biopolymers dissolve.

Protein and peptide pharmaceuticals can also be delivered orally. The process employs a natural process for oral uptake of vitamin B₁₂ in the body to co-deliver proteins and peptides. By riding the vitamin B₁₂ uptake system, the protein or peptide can move through the intestinal wall. Complexes are synthesised between vitamin B₁₂ analogues and the drug that retain both significant affinity for intrinsic factor (IF) in the vitamin B₁₂ portion of the complex and significant bioactivity of the drug portion of the complex.

Proteins and polypeptides can be introduced to cells by “Trojan peptides”. These are a class of polypeptides called penetratins which have translocating properties and are capable of carrying hydrophilic compounds across the plasma membrane. This system allows direct targetting of oligopeptides to the cytoplasm and nucleus, and may be non-cell type specific and highly efficient. See Derossi et al (1998), Trends Cell Biol 8, 84-87.

It is preferred if the treatment agent (or agents) is administered orally. It is further preferred if the treatment agent (or agents) is administered to the female reproductive system. For example, the treatment agent or agents may suitably be administered intravaginally using, for example, a gel or cream or vaginal ring or tampon. The treatment agent may also advantageously be administered by intrauterine delivery, for example using methods well known in the art such as an intrauterine device.

Typically, the gel or cream is one which is formulated for administration to the vagina. It may be oil based or water based. Typically, the treatment agent (or agents) is present in the cream or gel in a sufficient concentration so that an effective amount is administered in a single (or in repeated) application.

Typically, the vaginal ring comprises a polymer which formed into a “doughnut” shape which fits within the vagina. The treatment agent (or agents) is present within the polymer, typically as a core, which may dissipate through the polymer and into the vagina and/or cervix in a controlled fashion. Vaginal rings are known in the art.

Typically, the tampon is impregnated with the treatment agent (or agents) and that a sufficient amount of the treatment agent (or agents) is present in the tampon.

Typically, the intrauterine device is for placing in the uterus over extended periods of time, such as between one and five years. Typically, the intrauterine device comprises a plastic frame, often in the shape of a “T” and contains sufficient antagonist to be released over the period of use. The antagonist is generally present within or encompassed by a slow-release polymer which forms part of the device, such as in the form of a “sausage” of antagonist which wraps around the long arm of the “T” which is typically covered with a controlled-release membrane. Intrauterine devices are known in the art.

The individual to be treated may be any female individual who would benefit from such treatment. Typically and preferably the individual to be treated is a human female. However, the methods of the invention may be used to treat female mammals, such as the females of the following species: cows, horses, pigs, sheep, cats and dogs. Thus, the methods have uses in both human and veterinary medicine.

A second aspect of the invention provides use of any one or more of an inhibitor of cyclooxygenase-2 (COX-2), an inhibitor of prostaglandin E synthase (PGES), or an EP2 or EP4 receptor antagonist in the manufacture of a medicament for treating or preventing a pathological condition of the uterus.

The invention will now be described in more detail by reference to the following Examples and Figures wherein:

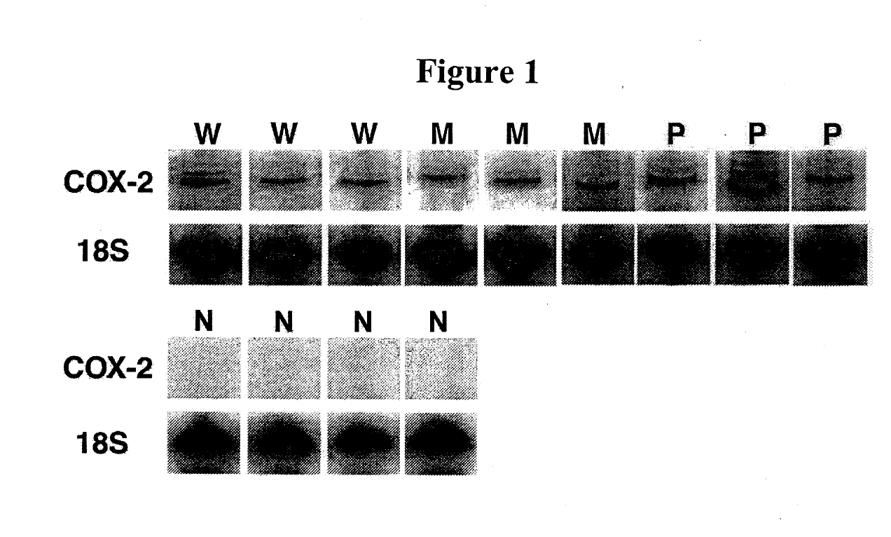

FIG. 1. Ribonuclease protection assay conducted using 10 Tg of total RNA extracted from normal secretory phase endometrium (N) and well (W), moderately (M) and poorly (P) differentiated endometrial adenocarcinoma tissue. COX-2 expression was detected using a 381 homologous cRNA probe. The integrity of the RNA and the relative amount of total RNA in each reaction were determined using a ribosomal 18S cDNA probe.

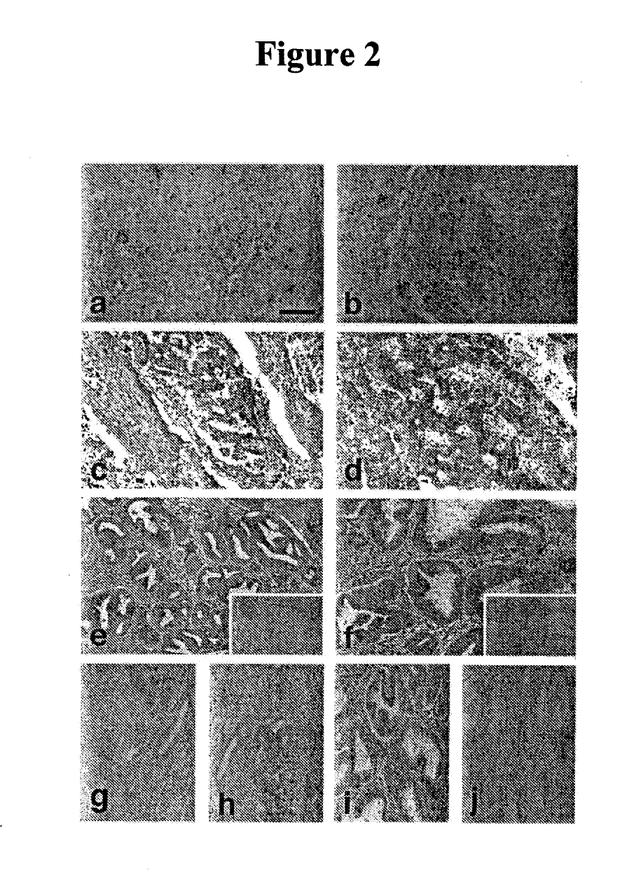

FIG. 2. COX-2 expression and PGE₂ synthesis are detected in epithelial cells of poorly (Figure a and b respectively), moderately (Figure c and d respectively) and well (Figure e and f respectively) differentiated endometrial adenocarcinoma. Minimal immunostaining for COX2 or PGE₂ were detected in post menopausal (Figure g and h) or secretory phase (Figure i and j) endometrium. Insets in FIGS. 2e and 2f are sections that were stained with pre-adsorbed COX-2 and PGE₂ sera respectively (negative controls). Scale bar is 100 Tm.

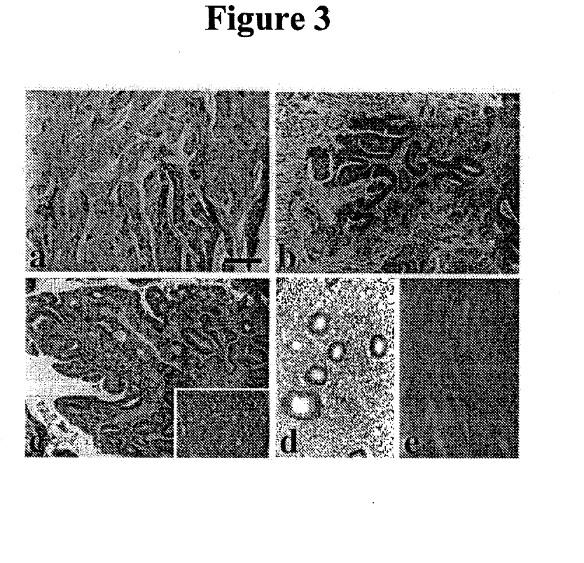

FIG. 3. mPGES expression is detected in epithelial cells of poorly (Figure a), moderately (Figure b) and well (Figure c) differentiated endometrial adenocarcinoma. Minimal immunostaining for mPGES was detected in post-menopausal uterus and secretory phase endometrium (Figure d and e respectively). Inset in Figure c is a section that was stained with mPGES pre-adsorbed serum (negative control). Scale bar is 100 Tm.

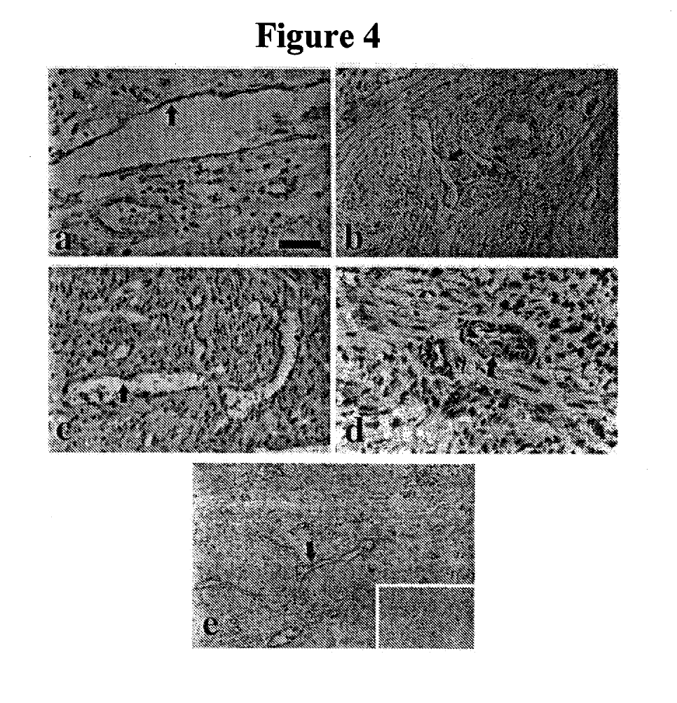

FIG. 4. COX-2 (Figure a), mPGES (Figure b), PGE₂ (Figure c) and EP4 (Figure d) are detected in endothelial cells of all carcinoma tissues. Vascular endothelial cells in endometrial adenocarcinoma were localised using antibodies raised against the human CD34 endothelial cell marker (Figure e). The inset in Figure e is a section that was stained with non-immune goat serum (CD34 negative control). Negative controls for the other antibodies are presented in FIGS. 2, 3 and 5B. Scale bar is 50 Tm.

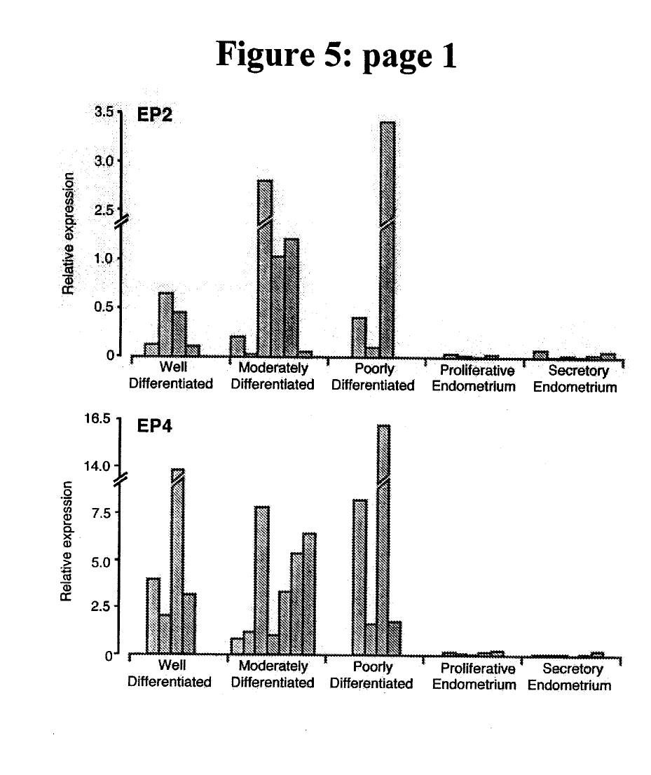

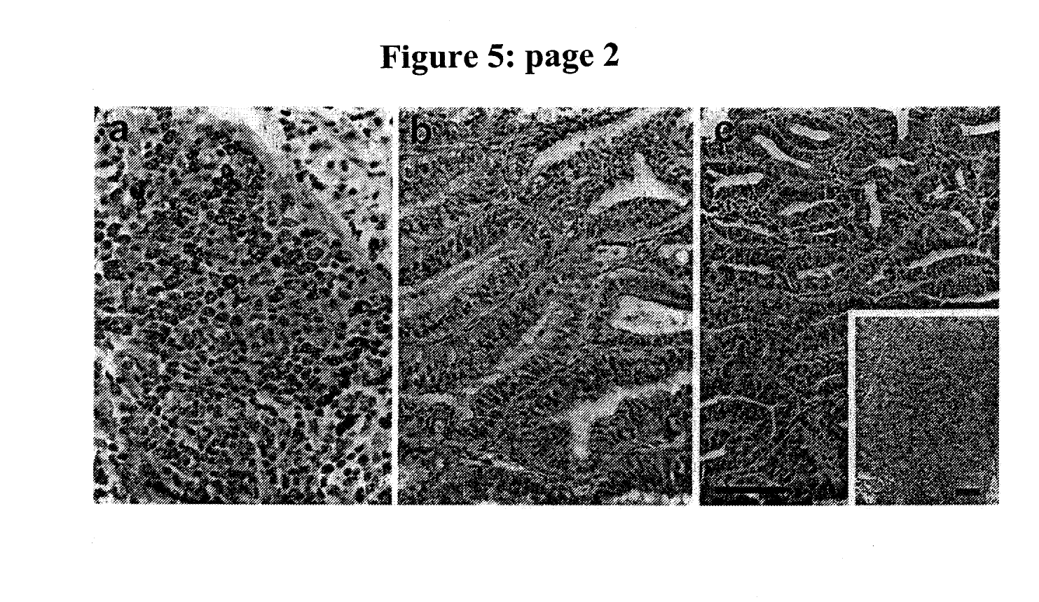

FIG. 5. A. Relative expression of EP2 and EP4 receptors in endometrial adenocarcinoma of different grades of differentiation and in healthy secretory phase endometrium collected from fertile women with normal menstrual cycles. B. EP4 receptor expression was detected in neoplastic epithelial cells of poorly (Figure a), moderately (Figure b) and well (Figure c) differentiated uterine adenocarcinomas. The inset in Figure c is a section that was stained with immune serum that had been pre-adsorbed with the blocking peptide. Scale bar is 50 Tm.

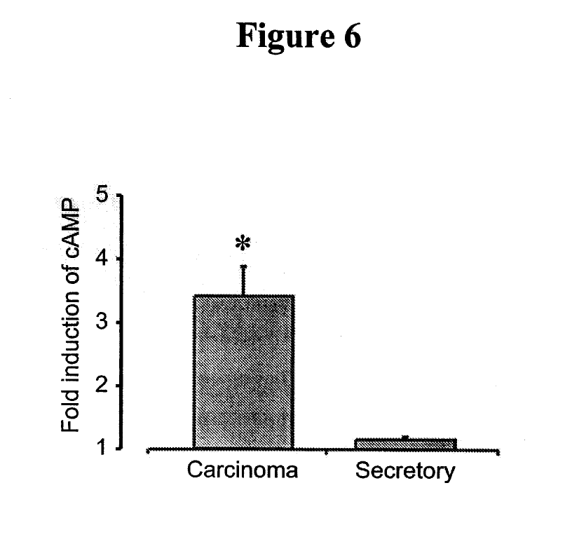

FIG. 6. Fold induction of cAMP response in endometrial adenocarcinoma (n=6) and healthy secretory phase endometrium (n=6) following stimulation with 300 nM PGE₂. Fold induction was calculated by dividing cAMP values for the PGE₂-treated samples by the values for the untreated sample.

### EXAMPLE 1

### Expression of COX-2 and PGE Synthase and Synthesis of PGE₂ in Endometrial Adenocarcinoma: a Possible Autocrine/Paracrine Regulation of Neoplastic Cell Function via EP2/EP4 Receptors

### Summary

This study was designed to investigate the possible role of cyclooxygenase-2 (COX-2) and prostaglandin E₂ (PGE₂) in endometrial adenocarcinoma. COX-2 RNA expression was confirmed in various grades of adenocarcinoma by ribonuclease protection assay. COX-2 and microsomal glutathione-dependent prostaglandin E synthase (mPGES) expression and PGE₂ synthesis were localised to the neoplastic epithelial cells and endothelial cells. In order to establish whether PGE₂ has an autocrine/paracrine effect in adenocarcinomas, we investigated the expression of two subtypes of PGE₂ receptors, namely EP2 and EP4, by real time quantitative PCR. Expression of EP2 and EP4 receptors was detected in adenocarcinomas from all grades of differentiation and was significantly higher than that detected in normal secretory phase endometrium (P<0.01). The fold induction of expression in adenocarcinoma compared with normal secretory phase endometrium was 28.0±7.4 and 52.5±10.1 for EP2 and EP4 receptors respectively. Immunohistochemistry localised the site of expression of EP4 receptor in neoplastic epithelial cells and in the endothelium of carcinomas of all grades of differentiation. Finally, the functionality of the EP2/EP4 receptors was assessed by investigating cAMP generation following in vitro culture of adenocarcinoma tissue in the presence or absence of 300 nM PGE₂. cAMP production in response to PGE₂ was significantly higher in carcinoma tissue than that detected in normal secretory phase endometrium (3.42±0.46 vs 1.15±0.05 respectively; P<0.001). In conclusion, these data suggest that PGE₂ may regulate neoplastic cell function in an autocrine/paracrine manner via the EP2/EP4 receptors.

### Materials and Methods

Tissue collection and Processing. Endometrial adenocarcinoma tissue was collected from women undergoing hysterectomy and who had been pre-diagnosed to have adenocarcinoma of the uterus. All women with endometrial adenocarcinoma were post-menopausal. To provide control tissue, normal secretory phase (Days 18-25 of the menstrual cycle) endometrial tissue was collected with a pipelle suction curette (Pipelle; Laboratoire CCD, Paris, France) from fertile women with regular menstrual cycles, undergoing gynecological procedures for benign conditions. Biopsies were dated from the patient's last menstrual period (LMP) and histological dating was consistent with date of LMP. Subjects had not been exposed to exogenous hormones for at least six months prior to inclusion in the study. This phase of the menstrual cycle was chosen for comparison with endometrial adenocarcinoma tissue as minimal proliferative activity of the endometrium is detected during the secretory phase (Ferenczy et al., 1979). This would be comparable to the absence of proliferative activity predicted in healthy post-menopausal endometrium. Shortly after hysterectomy or pipelle suction, the tissue was either snap frozen in dry ice and stored at −70° C. (for RNA extraction), fixed in Neutral buffered formalin and wax embedded (for immunohistochemical analyses) or placed in RPMI 1640 (containing 2 mmol/L L-glutamine, 100 U penicillin and 100 μg/mL streptomycin) and transported to the laboratory for in vitro culture. In addition, archival tissue blocks of healthy post-menopausal uterus were obtained from The Department of Pathology (The University of Edinburgh Medical School) and utilised for immunohistochemical analyses. Written informed consent was obtained prior to tissue collection and ethical approval was received from the Lothian Research Ethics Committee. The data in this study were analysed by ANOVA using StatView 5.0.

Ribonuclease Protection assay. RNA was extracted from endometrial adenocarcinoma tissue (n=3 of each of well, moderately or poorly differentiated endometrial adenocarcinoma) and normal secretory phase endometrium (n=4) using Tri-Reagent as recommended by the manufacturer (Sigma, Dorset, UK). A homologous 381 bp COX-2 cDNA probe was generated by PCR from a clone containing the human COX-2 cDNA (the clone was a gift from Dr S Prescott, University of Utah) and primers at base pair position 950-971 (COX2A: 5′-CAAGCAGGCTAATACTGATAGG-3′) (SEQ ID NO: 9) and 1310-1331 (COX2B: 5′-ATCTGCCTGCTCTGGTCAATGG-3′) (SEQ ID NO: 10). The amplified PCR product was subcloned into pCRII and its identity and orientation confirmed following sequencing using the Applied Biosystems 373A DNA sequencer and the ABI prism DNA sequencing kit (Applied Biosystems, Cheshire, UK).

For the RPA, an antisense cRNA probe was prepared from HindIII linearised pCRII plasmid containing the 381 bp cDNA fragment of the human COX-2. The RPA was conducted using the Ambion RPA II kit (AMS Biotechnology Europe, Oxfordshire, UK) as previously reported (Jabbour et al., 1998). Briefly, radiolabelled cRNA was generated using the linearised plasmid, T7 RNA polymerase and I³²P-UTP (800 Ci/mmol; Amersham, Buckinghamshire, UK). Total RNA (10 Tg) from adenocarcinoma tissue and yeast (n=2; used as reaction controls in the presence or absence of RNase digestion to establish the specificity of the hybridisation reaction and the size of the unprotected RNA fragment) was mixed with the radiolabelled probe (2×10⁵ cpm) and hybridisation buffer, heated to 90° C. for 4 min and incubated overnight at 45° C. Integrity of RNA and the relative amount of total RNA in each reaction was determined by including 18S radiolabelled cRNA in each reaction. Single stranded RNA were digested using 250 U/ml RNase A and 10000 U/ml RNase T1 for 30 min at 37° C. The protected RNA was precipitated and separated on a 5% denaturing acrylamide gel. The gel was dried under vacuum and exposed to an autoradiographic film (XAR-5; Kodak).

Immunohistochemistry. Immunohistochemistry was performed on adenocarcinoma tissue (n=4 of each of well, moderately and poorly differentiated), normal secretory phase endometrium (n=4) and healthy post-menopausal uterus (n=4). Five-micron paraffin wax-embedded tissue sections were dewaxed in xylene, rehydrated in graded ethanol and washed in water followed by TBS (50 mM Tris-HCl, 150 mM NaCl pH 7.4) and blocked for endogenous peroxidase (3% H₂O₂ in methanol). Sections were blocked using either 20% normal rabbit serum (for COX-2), 20% swine serum (for mPGES, PGE₂ and EP4) or 20% normal goat serum (for CD34) diluted in TBS. Subsequently the tissue sections were incubated with polyclonal goat anti-COX-2 antibody (sc-1745; Autogenbioclear, Wilts, UK) at a dilution of 1:400, polyclonal rabbit anti-mPGES antibody (catalogue number 160140; Cayman Chemical, Alexis Corporation-Europe, Nottingham, UK) at a dilution of 1:50, polyclonal rabbit anti-EP4 receptor (catalogue number 101770; Cayman Chemical) at a dilution of 1:500, polyclonal rabbit anti-PGE₂ antibody (kindly supplied by Professor R W Kelly, MRC Human Reproductive Sciences Unit, Edinburgh, UK) at a dilution of 1:100 or monoclonal mouse anti-human CD34 primary antibody (mca-547; Serotec, Oxford, UK) at a dilution of 1:25 at 4° C. for 18 h. Control tissue was incubated with either 5% non-immune antisera (CD34), goat anti-COX-2 antibody pre-adsorbed to blocking peptide (sc-1745p; Autogenbioclear), rabbit anti-mPGES antibody pre-adsorbed to blocking peptide (catalogue number 360140; Cayman Chemical), rabbit anti-EP4 pre-adsorbed to blocking peptide (catalogue number 101780; Caman Chemical) or rabbit anti-PGE₂ antibody pre-adsorbed to 10-fold excess PGE₂ (Sigma). After thorough washing with TBS, the tissue sections probed with the goat anti-human COX-2, rabbit anti-mPGES, rabbit anti-EP4 and rabbit anti-PGE₂ primary antibodies were incubated with biotinylated rabbit anti-goat secondary IgG antibody (for COX-2; Dako, Bucks, UK) or swine anti-rabbit secondary IgG antibody (for mPGES, EP4 and PGE₂; Dako) at a dilution of 1:500 for 40 min at 25° C. Thereafter the tissue sections were incubated with streptavidin-biotin peroxidase complex (Dako) for 20 min at 25° C. Tissue sections probed with the mouse anti-human CD34 antibody were developed using a Mouse EnVision Kit (Dako) as instructed by the manufacturer. Colour reaction was developed by incubation with 3.3′-diaminobenzidine (Dako).

Real time quantitative PCR. Endometrial RNA samples were extracted from adenocarcinoma tissue (n=4 well differentiated, n=6 moderately differentiated, n=4 poorly differentiated) and normal secretory phase endometrium (n=7) as described above. RNA samples were reverse transcribed using MgCl₂ (5.5 mM), dNTPs (0.5 mM each), random hexamers (2.5 μM), RNAase inhibitor (0.4 U/μl) and multiscribe reverse transcriptase (1.25 U/μl; all from PE Biosystems, Warrington, UK). The mix was aliquoted into individual tubes (16 μl/tube) and template RNA was added (4 μl/tube of 100 ng/μl RNA). Samples were incubated for 60 minutes at 25° C., 45 minutes at 48° C. and then at 95° C. for 5 minutes.

A reaction mix was made containing Taqman buffer (5.5 mM MgCl₂, 200 μM dATP, 200 μM dCTP, 200 μM dGTP, 400 μM dUTP), ribosomal 18S forward and reverse primers and probe (all at 50 nM), forward and reverse primers for EP receptor (300 nM), EP receptor probe (200 nM), AmpErase UNG (0.01 U/μl) and AmpliTaq Gold DNA Polymerase (0.025 U/μl; all from PE Biosystems). A volume of 48 μl of reaction mix was aliquoted into separate tubes for each cDNA sample and 2 μl/replicate of cDNA was added. After mixing, 23 μl of sample were added to the wells on a PCR plate. Each sample was added in duplicate. A no template control (containing water) was included in triplicate. Wells were sealed with optical caps and the PCR reaction run on an ABI Prism 7700 using standard conditions. EP receptor primers and probe for quantitative PCR were designed using the PRIMER express program (PE Biosystems). The sequence of the EP2 receptor primers and probe were as follows; Forward: 5′-GAC CGC TTA CCT GCA GCT GTA C-3′ (SEQ ID NO: 11); Reverse: 5′-TGA AGT TGC AGG CGA GCA-3′ (SEQ ID NO: 12); Probe (FAM labelled): 5′-CCA CCC TGC TGC TGC TTC TCA TTG TCT-3′ (SEQ ID NO: 13). The sequence of the EP4 receptor primers and probe were as follows; Forward: 5′-ACG CCG CCT ACT CCT ACA TG-3′ (SEQ ID NO: 14); Reverse: 5′-AGA GGA CGG TGG CGA GAA T-3′ (SEQ ID NO: 15); Probe (FAM labelled): 5′-ACG CGG GCT TCA GCT CCT TCC T-3′ (SEQ ID NO: 16). The ribosomal 18S primers and probe sequences were as follows; Forward: 5′-CGG CTA CCA CAT CCA AGG AA-3′ (SEQ ID NO: 17); Reverse: 5′-GCT GGA ATT ACC GCG GCT-3′ (SEQ ID NO: 18); Probe (VIC labelled): 5′-TGC TGG CAC CAG ACT TGC CCT C-3′ (SEQ ID NO: 19).

In vitro culture and cAMP measurement. Endometrial tumour tissue (n=6; two well, two moderately and two poorly differentiated adenocarcinomas) and normal secretory phase endometrium (n=6) were minced finely with scissors and incubated in 2 ml RPMI (Sigma) medium containing 10% fetal calf serum, 0.3 mg/ml L-glutamine, 100 IU penicillin, 100 μg streptomycin and 3 μg/ml indomethacin, for 1.5 hrs at 37C in humidified 5% CO₂. Thereafter samples were incubated in the same medium containing IBMX (Sigma) to a final concentration of 1 mM for 30 min at 37C and then stimulated for 5 min with 300 nM PGE₂. Control treatments received no PGE₂. Tissue was harvested by centrifugation at 2000 g. The supernatant was discarded and the tissue homogenised in 0.1 M HCl. cAMP concentration was quantified by ELISA using a cAMP kit (Biomol; Affiniti, Exeter, UK) and normalised to protein concentration of the homogenate. Protein concentrations were determined using protein assay kits (Bio-Rad, Hemel Hempstead, UK). The data are presented as fold induction of cAMP following treatment with PGE₂. Fold induction was calculated by dividing cAMP values for the PGE₂-treated samples by the value for the untreated sample.

### Results

COX-2 expression in endometrial adenocarcinoma was assessed by ribonuclease protection assay. COX-2 expression was detected in well, moderately and poorly differentiated (n=3 each grade) adenocarcinomas (FIG. 1). No COX-2 expression was detected in the secretory phase endometrium collected from women with normal menstrual cycles (FIG. 1). It was not possible to obtain fresh healthy post-menopausal endometrium to assess COX-2 RNA expression and hence secretory phase endometrium was chosen as a comparative tissue for the adenocarcinoma. This phase of the menstrual cycle was chosen as minimal proliferative activity is detected in the endometrium during the secretory phase (Ferenczy et al., 1979). This would be comparable to the absence of proliferative activity predicted in healthy post-menopausal endometrium.

Immunohistochemistry was employed in order to detect the site of expression of COX-2/mPGES and synthesis of PGE₂ in the endometrial adenocarcinomas. COX-2 and mPGES expression was detected in neoplastic epithelial cells in poorly, moderately and well differentiated adenocarcinoma (FIGS. 2a, 2c and 2e for COX-2 and FIGS. 3a, 3b and 3c for mPGES respectively). Minimal signal was detected in archival post-menopausal uterine specimens or secretory phase endometrium (FIGS. 2g, 2i, 3d and 3e) and no signal was detected when the antibody was pre-adsorbed with blocking peptide (FIGS. 2e and 3c insets). Similar to COX-2/mPGES, PGE₂ synthesis was detected in the neoplastic epithelial cells of carcinomas of different grades of differentiation (FIGS. 2b, 2d and 2f respectively). Minimal immunostaining for PGE₂ was observed in post-menopausal uterus or secretory phase endometrium (FIGS. 2h and 2j respectively) and no staining was detected in tissue treated with pre-adsorbed sera (FIG. 2f inset). In addition, COX-2, mPGES and PGE₂ immunostaining was observed in endothelial cells lining the vasculature in all adenocarcinoma sections investigated (FIGS. 4a, 4b and 4c respectively). To confirm that COX-2/mPGES expression and PGE₂ synthesis were localised to the endothelial cells of blood vessels, immunohistochemistry was performed on tissue sections using antibodies raised against the CD34 endothelial cell marker. The pattern of expression with CD34 (FIG. 4e) was identical to that observed with COX-2, mPGES and PGE₂ thus confirming that COX-2/mPGES expression and PGE₂ synthesis are localised to the endothelial cell layer of blood vessels in human adenocarcinomas. Negligible staining was observed in the stromal compartment of all carcinoma tissue investigated.

The expression of two subtypes of PGE₂ receptors, namely EP2 and EP4, was investigated by real-time quantitative PCR in carcinoma tissue and normal secretory phase endometrium. Expression of both receptors was significantly up-regulated in adenocarcinoma tissues compared with normal secretory endometrium (P<0.01). No differences in the level of expression of EP2 or EP4 receptors were detected between poorly, moderately or well differentiated adenocarcinomas (FIG. 5A). Overall, the fold induction of EP2 and EP4 receptor expression in adenocarcinoma tissue (mean fold induction of all carcinoma samples) compared with normal secretory endometrium was 28.0±7.4 and 52.5±10.1 for EP2 and EP4 receptors respectively. Using immunohistochemistry, EP4 receptor expression was localised to neoplastic epithelial cells of carcinoma tissues of all grades of differentiation (FIG. 5B) and also in endothelial cells of the microvasculature (FIG. 4d).

In order to assess the activity of the EP2/EP4 receptors in the carcinoma tissue and normal secretory phase endometrium, cAMP generation was measured following short term in vitro culture with or without PGE₂ (FIG. 6). Comparable cAMP turnover in response to PGE₂ was observed in all carcinoma tissue. The fold induction of cAMP generation in response to PGE₂ was significantly higher in carcinoma tissue compared with secretory phase endometrium (3.42±0.46 vs 1.15±0.05 respectively; P<0.001).

### Discussion

The data presented in this study demonstrate the expression of COX-2 and mPGES enzymes in adenocarcinomas of the uterus at different grades of differentiation as demonstrated by ribonuclease protection assays and immunohistochemistry. COX-2 and mPGES expression were co-localised to neoplastic epithelial cells and endothelial cells of the microvasculature suggesting a co-regulated pattern of expression for the two genes. Previous studies have detected expression of mPGES in human smooth muscle vascular cells but not in umbilical vein endothelial cells although these cells retained synthetic capacity for PGE₂ (Soler et al., 2000). This apparent discrepancy may reflect tissue variation in regulation of expression of mPGES in endothelial cells. The over-expression of COX-2 enzyme observed in endometrial adenocarcinomas resembles that reported for a number of other carcinomas including colon, lung, bladder, stomach, pancreas, prostate and cervix (Gupta et al., 2000; Mohammed et al., 1999; Ratnasinghe et al., 1999; Sales et al., 2001; Tsujii et al., 1997; Tucker et al., 1999; Wolff et al., 1998). The exact intracellular signalling pathways that lead to up-regulation in COX-2 expression in carcinomas remain to be elucidated. However, recent data suggest regulatory roles for ERK2 MAP kinase (Jones et al., 1999a), p38 MAP kinase (Dean et al., 1999) and Phosphatidylinositol 3-kinase (Weaver et al., 2001) in a number of model systems including endometrial adenocarcinoma epithelial cells (Munir et al., 2000).

The immunohistochemistry studies suggest that COX-2 and mPGES expression is associated with enhanced production of PGE₂ in neoplastic cells and endothelial cells of the microvasculature. Previous studies have shown that PGE₂ synthesis/secretion is significantly elevated in uterine carcinomas compared with normal uterus (Willman et al., 1976). Moreover, in a number of model systems PGE₂ synthesis and secretion are elevated in response to COX-2 up-regulation (Tsujii & DuBois, 1995). The biological role of COX-2 and PGE₂ in endometrial adenocarcinomas remains to be established. However, enhanced COX-2 expression and PGE₂ synthesis can induce neoplastic changes in epithelial cells through a number of biological pathways. These include promotion of cellular proliferation, inhibition of apoptosis, increasing metastatic potential of neoplastic cells and promoting angiogenesis (Rolland et al., 1980; Tsuji et al., 1996; Tsujii & DuBois, 1995). Over-expression of COX-2 enzyme in rat intestinal epithelial cells results in enhanced secretion of PGE₂ which is associated with increased cellular proliferation and resistance to apoptosis (Tsujii & DuBois, 1995). Similarly, over expression of COX-2 and increased production of prostaglandins have been linked to enhanced metastatic potential of neoplastic cells possibly through down regulation of expression of cell adhesion molecules such as E-Cadherin (Rolland et al., 1980). Expression of E-Cadherin is down regulated in a number of solid tumours and is closely and inversely related to enhanced invasion of neoplastically transformed cells (Mayer et al., 1993; Schipper et al., 1991).

Successful tumour establishment and metastasis is also dependent on initiation of angiogenesis at the site of growth of the tumour cells. COX-2 and PGE₂ are strongly linked with regulation of the angiogenic process during tumour development (Masferrer et al., 2000). Over-expression of COX-2 and increased production of PGE₂ in epithelial cells enhances the expression of angiogenic factors which act in a paracrine manner to induce endothelial cell migration and microvascular tube formation (Tsujii et al., 1998). Similarly, COX-2 and PGE₂ may influence angiogenesis directly by acting on endothelial cells. Treatment of endothelial cells with selective COX-2 inhibitors has been shown to reduce microvascular tube formation and this effect is partially reversed by co-treatment with PGE₂ (Jones et al., 1999b). Hence, it is feasible that in vivo angiogenesis in endometrial adenocarcinomas may be regulated by COX-2 and PGE₂ via an epithelial-endothelial and an endothelial-endothelial cell interaction. This is supported by the data presented in this study which localised the site of expression of COX-2, mPGES and PGE₂ to neoplastic epithelial cells and endothelial cells.

PGE₂ acts on target cells through interaction with seven transmembrane G-protein coupled receptors. Different forms of the membrane bound receptors have been cloned which utilise alternate intracellular signalling pathways. In this study we investigated the expression of two of the membrane bound PGE₂ receptors, namely EP2 and EP4, which mediate their effect on target cells via the PKA pathway by activating adenylate cyclase and increasing intracellular cAMP (Coleman et al., 1994). In endometrial adenocarcinoma, expression of EP2 and EP4 receptors is up-regulated in comparison with normal secretory phase endometrium and expression of at least the EP4 receptor is localised to neoplastic epithelial cells and the endothelium of the microvasculature. It was not possible to conduct parallel studies to localise EP2 receptors in the carcinoma tissues as no commercial antibodies are available for this receptor. Hence it remains to be established whether these receptors are co-expressed in the same cell type. However, using in situ hybridisation techniques, EP2 and EP4 receptor expression have been recently co-localised to epithelial and endothelial cells of the normal human endometrium (Milne et al., 2001). Functionality of the EP2/EP4 receptors in the carcinoma tissue was assessed by measuring cAMP generation following treatment with exogenous PGE₂. Treatment with PGE₂ resulted in a rapid cAMP generation thus demonstrating functional activation of the EP2 and/or EP4 receptors in this tissue. Up-regulation in expression and signalling of EP2/EP4 receptors has also been reported in cervical carcinoma and this suggests a common signalling pathway for PGE₂ in reproductive tract neoplasia (Sales et al., 2001). The exact role of COX-2/mPGES enzymes and PGE₂ and the associated EP2/EP4 receptors in endometrial adenocarcinoma remains to be established. However, it is reasonable to suggest that COX-2/mPGES and PGE₂ may mediate proliferation of epithelial and/or endothelial cells. The proliferating cells within endometrial adenocarcinomas are detected predominantly in post-menopausal women at a time when the healthy endometrium is expected to atrophy and display minimal cellular proliferation or angiogenesis. Moreover, healthy post-menopausal uterus and normal secretory endometrium, both of which have minimal proliferative or angiogenic activity, display negligible COX-2/mPGES/EP2/EP4 receptor expression and minimal cAMP generation in response to treatment with PGE₂. A possible role for PGE₂ in proliferation has already been established in a number of cell types including endothelial cells and it has been suggested that this effect is mediated via cAMP and induction of expression of mitogenic growth factors such as vascular endothelial growth factor and basic fibroblast growth factor (Cheng et al., 1998; Hoper et al., 1997). Future studies will elucidate the exact role of PGE₂ and its associated receptors on proliferation and neoplastic differentiation of epithelial/endothelial cells in endometrial adenocarcinomas.

In conclusion, these data confirm the expression of COX-2 and mPGES enzymes and synthesis of PGE₂ in endometrial adenocarcinoma of various grades of differentiation. Both COX-2/mPGES and PGE₂ are localised to the neoplastic epithelial cells and endothelial cells of the microvasculature. PGE₂ may exert an autocrine/paracrine effect in endometrial adenocarcinoma through interaction with EP2/EP4 receptors and activation of the PKA signalling pathway.

### EXAMPLE 2

### Treatment of Uterine Cancer with a COX-2 Inhibitor

A patient suffering from uterine cancer is administered meloxicam.

### EXAMPLE 3

### Treatment of Fibroids with EP4 Receptor Antagonist

A patient suffering from fibroids is administered AH23848B.

### EXAMPLE 4

### Treatment of Endometriosis with COX-2 Inhibitor

A patient suffering from endometriosis is administered nimesulide.

### REFERENCES FOR EXAMPLE 1

Ashby, B. (1998) Co-expression of prostaglandin receptors with opposite effects: a model for homeostatic control of autocrine and paracrine signaling. Biochem Pharmacol 55: 239-246.
Cheng, T, Cao, W, Wen, R, Steinberg, R H, LaVail, M M. (1998) Prostaglandin E₂ induces vascular endothelial growth factor and basic fibroblast growth factor mRNA expression in cultured rat Muller cells. Invest Ophthalmol Vis Sci 39: 581-591.
Coleman, R A, Smith, W L, Narumiya, S. (1994) International Union of Pharmacology classification of prostanoid receptors: properties, distribution, and structure of the receptors and their subtypes. Pharmacol Rev 46: 205-229.
Dean, J L, Brook, M, Clark, A R, Saklatvala, J. (1999) p38 mitogen-activated protein kinase regulates cyclooxygenase-2 mRNA stability and transcription in lipopolysaccharide-treated human monocytes. J Biol Chem 274: 264-269.
DeWitt, D L. (1991) Prostaglandin endoperoxide synthase: regulation of enzyme expression. Biochim Biophys Acta 1083: 121-134.
Ferenczy, A, Bertrand, G, Gelfand, M M. (1979) Proliferation kinetics of human endometrium during the normal menstrual cycle. Am J Obstet Gynecol 133: 859-867.
Gordon, M D, Ireland, K. (1994) Pathology of hyperplasia and carcinoma of the endometrium. Semin Oncol 21: 64-70.
Gupta, S, Srivastava, M, Ahmad, N, Bostwick, D G, Mukhtar, H. (2000) Over-expression of cyclooxygenase-2 in human prostate adenocarcinoma. Prostate 42: 73-78.
Herschman, H R. (1996) Prostaglandin synthase 2. Biochim Biophys Acta 1299: 125-140.
Hoper, M M, Voelkel, N F, Bates, T O, Allard, J D, Horan, M, Shepherd, D, Tuder, R M. (1997) Prostaglandins induce vascular endothelial growth factor in a human monocytic cell line and rat lungs via cAMP. Am J Respir Cell Mol Biol 17: 748-756.
Jabbour, H N, Critchley, H O D, Boddy, S C. (1998) Expression of functional prolactin receptors in non-pregnant human endometrium: Janus kinase-2, signal transducer and activator of transcription-1 (STAT1), and STAT5 proteins are phosphorylated after stimulation with prolactin. Journal of Clinical Endocrinology and Metabolism 83: 2545-2553.
Jakobsson, P J, Thoren, S, Morgenstern, R, Samuelsson, B. (1999) Identification of human prostaglandin E synthase: a microsomal, glutathione-dependent, inducible enzyme, constituting a potential novel drug target. Proc Natl Acad Sci USA 96: 7220-7225.
Jones, M K, Sasaki, E, Halter, F, Pai, R, Nakamura, T, Arakawa, T, Kuroki, T, Tarnawski, A S. (1999a) HGF triggers activation of the COX-2 gene in rat gastric epithelial cells: action mediated through the ERK2 signaling pathway. Faseb J 13: 2186-2194.
Jones, M K, Wang, H, Peskar, B M, Levin, E, Itani, R M, Sarfeh, I J, Tarnawski, A S. (1999b) Inhibition of angiogenesis by nonsteroidal anti-inflammatory drugs: insight into mechanisms and implications for cancer growth and ulcer healing. Nat Med 5: 1418-1423.
Mant, J W, Vessey, M P. (1994) Ovarian and endometrial cancers. Cancer Surv 20: 287-307.
Masferrer, J L, Leahy, K M, Koki, A T, Zweifel, B S, Settle, S L, Woerner, B M, Edwards, D A, Flickinger, A G, Moore, R J, Seibert, K. (2000) Antiangiogenic and antitumor activities of cyclooxygenase-2 inhibitors. Cancer Res 60: 1306-1311.
Mayer, B, Johnson, J P, Leitl, F, Jauch, K W, Heiss, M M, Schildberg, F W, Birchmeier, W, Funke, I. (1993) E-cadherin expression in primary and metastatic gastric cancer: down-regulation correlates with cellular dedifferentiation and glandular disintegration. Cancer Res 53: 1690-1695.
Milne, S A, Perchick, G B, Boddy, S C, Jabbour, H N (2001) Expression, localisation and signalling of PGE₂ and EP2/EP4 receptors in human non-pregnant endometrium across the menstrual cycle. Journal of Clin Endocrinol Metab (in press).
Mohammed, S I, Knapp, D W, Bostwick, D G, Foster, R S, Khan, K N, Masferrer, J L, Woerner, B M, Snyder, P W, Koki, A T. (1999) Expression of cyclooxygenase-2 (COX-2) in human invasive transitional cell carcinoma (TCC) of the urinary bladder. Cancer Res 59: 5647-5650.
Munir, I, Fukunaga, K, Kanasaki, H, Miyazaki, K, Ohba, T, Okamura, H, Miyamoto, E. (2000) Expression of cyclooxygenase 2 by prostaglandin E₂ in human endometrial adenocarcinoma cell line HEC-1B. Biol Reprod 63: 933-941.
Ratnasinghe, D, Tangrea, J A, Roth, M J, Dawsey, S M, Anver, M, Kasprzak, B A, Hu, N, Wang, Q H, Taylor, P R. (1999) Expression of cyclooxygenase-2 in human adenocarcinomas of the gastric cardia and corpus. Oncol Rep 6: 965-968.
Rolland, P H, Martin, P M, Jacquemier, J, Rolland, A M, Toga, M. (1980) Prostaglandin in human breast cancer: Evidence suggesting that an elevated prostaglandin production is a marker of high metastatic potential for neoplastic cells. J Natl Cancer Inst 64: 1061-1070.
Sales, K J, Katz, A A, Davis, M, Hinz, S, Soeters, R P, Hofmeyr, M D, Millar, R P, Jabbour, H N. (2001) Cyclooxygenase-2 Expression and Prostaglandin E₂ Synthesis Are Up-Regulated in Carcinomas of the Cervix: A Possible Autocrine/Paracrine Regulation of Neoplastic Cell Function via EP2/EP4 Receptors. J Clin Endocrinol Metab 86: 2243-2249.
Schipper, J H, Frixen, U H, Behrens, J, Unger, A, Jahnke, K, Birchmeier, W. (1991) E-cadherin expression in squamous cell carcinomas of head and neck: inverse correlation with tumor dedifferentiation and lymph node metastasis. Cancer Res 51: 6328-6337.
Soler, M, Camacho, M, Escudero, J R, Iniguez, M A, Vila, L. (2000) Human vascular smooth muscle cells but not endothelial cells express prostaglandin E synthase. Circ Res 87: 504-507.
Subbaramaiah, K, Telang, N, Ramonetti, J T, Araki, R, DeVito, B, Weksler, B B, Dannenberg, A J. (1996) Transcription of cyclooxygenase-2 is enhanced in transformed mammary epithelial cells. Cancer Res 56: 4424-4429.
Tanioka, T, Nakatani, Y, Semmyo, N, Murakami, M, Kudo, I. (2000) Molecular identification of cytosolic prostaglandin E₂ synthase that is functionally coupled with cyclooxygenase-1 in immediate prostaglandin E₂ biosynthesis. J Biol Chem 275: 32775-32782.
Tsuji, S, Kawano, S, Sawaoka, H, Takei, Y, Kobayashi, I, Nagano, K, Fusamoto, H, Kamada, T. (1996) Evidences for involvement of cyclooxygenase-2 in proliferation of two gastrointestinal cancer cell lines. Prostaglandins Leukot Essent Fatty Acids 55: 179-183.
Tsujii, M, DuBois, R N. (1995) Alterations in cellular adhesion and apoptosis in epithelial cells overexpressing prostaglandin endoperoxide synthase 2. Cell 83: 493-501.
Tsujii, M, Kawano, S, DuBois, R N. (1997) Cyclooxygenase-2 expression in human colon cancer cells increases metastatic potential. Proc Natl Acad Sci USA 94: 3336-3340.
Tsujii, M, Kawano, S, Tsuji, S, Sawaoka, H, Hori, M, DuBois, R N. (1998) Cyclooxygenase regulates angiogenesis induced by colon cancer cells. Cell 93: 705-716.
Tucker, O N, Dannenberg, A J, Yang, E K, Zhang, F, Teng, L, Daly, J M, Soslow, R A, Masferrer, J L, Woerner, B M, Koki, A T, Fahey, T J, 3rd. (1999) Cyclooxygenase-2 expression is up-regulated in human pancreatic cancer. Cancer Res 59: 987-990.
Weaver, S A, Russo, M P, Wright, K L, Kolios, G, Jobin, C, Robertson, D A, Ward, S G. (2001) Regulatory role of phosphatidylinositol 3-kinase on TNF-alpha-induced cyclooxygenase 2 expression in colonic epithelial cells. Gastroenterology 120: 1117-1127.
Willman, E A, Collins, W P, Clayton, S G. (1976) Studies in the involvement of prostaglandins in uterine symptomatology and pathology. Br J Obstet Gynaecol 83: 337-341.
Wolff, H, Saukkonen, K, Anttila, S, Karjalainen, A, Vainio, H, Ristimaki, A. (1998) Expression of cyclooxygenase-2 in human lung carcinoma. Cancer Res 58: 4997-5001.

Each of the above-identified references is hereby incorporated by reference in its entirety.

1. A method of treating a pathological condition of the uterus in an individual the method comprising administering to the individual an EP2 or EP4 receptor antagonist, wherein the pathological condition of the 5 uterus is associated with abnormal growth of cells of the myometrium or endometrium.

2. A method according to claim 1 wherein the pathological condition of the uterus is endometriosis or myometrial fibroids.

3. A method according to claim 1 wherein an EP2 receptor antagonist is administered to the individual.

4. A method according to claim 3 wherein the EP2 receptor antagonist is AH6809.

5. A method according to claim 1 wherein an EP4 receptor antagonist is administered to the individual.

6. A method according to claim 5 wherein the EP4 receptor antagonist is any one or more of AH23848B, AH22921X, IFTSYLECL, IFASYECL, IFTSAECL, IFTSYEAL, ILASYECL, IFTSTDCL, XTSYEAL (where X is 4-biphenylalanine), XTSYEAL (where X is homophenyl alanine), and 5-thia-prostaglandin E derivatives described in WO 00/03980 (Ono Pharm Co Ltd), 5-butyl-2,4-dihydro-4-[[2′-[N-(3-chloro-2-thiophenecarbonyl)sulfamoyl]biphenyl-4-yl]methyl]-2-{2-(trifluoromethyl)phenyl]-1,2,4-triazol-3-one potassium salt, 5-butyl-2,4-dihydro-4-[[2′-[N-(2-methyl-3-furoyl)sulfamoyl]biphenyl-4-yl]methyl]-2-{2-(trifluoromethyl)phenyl]-1,2,4-triazol-3-one, 5-butyl-2,4-dihydro-4-[[2′-[N-(3-methyl-2-thiophenecarbonyl)sulfamoyl]biphenyl-4-yl]methyl]-2-{2-(trifluoromethyl)phenyl]-1,2,4-triazol-3-one, 5-butyl-2,4-dihydro-4-[[2′-[N-(2-thiophenecarbonyl)sulfamoyl]biphenyl-4-yl]methyl]-2-{2-(trifluoromethyl)phenyl]-1,2,4-triazol-3-one, and 5-butyl-2,4-dihydro-4-[[2′-[N-[2-(methypyrrole)carbonyl]sulfamoyl]biphenyl-4-yl]methyl]-2-{2-(trifluoromethyl)phenyl]-1,2,4-triazol-3-one.

7. A method of treating endometriosis in an individual the method comprising administering to the individual an EP2 or EP4 receptor antagonist under conditions effective to treat endometriosis.

8. A method according to claim 7 wherein an EP2 receptor antagonist is administered to the individual.

9. A method according to claim 8 wherein the EP2 receptor antagonist is AH6809.

10. A method according to claim 7 wherein an EP4 receptor antagonist is administered to the individual.

11. A method according to claim 10 wherein the EP4 receptor antagonist is any one or more of AH23848B, AH22921X, IFTSYLECL, IFASYECL, IFTSAECL, IFTSYEAL, ILASYECL, IFTSTDCL, XTSYEAL (where X is 4-biphenylalanine), XTSYEAL (where X is homophenyl alanine), and 5-thia-prostaglandin E derivatives described in WO 00/03980 (Ono Pharm Co Ltd), 5-butyl-2,4-dihydro-4-[[2′-[N-(3-chloro-2-thiophenecarbonyl)sulfamoyl]biphenyl-4-yl]methyl]-2-{2-(trifluoromethyl)phenyl]-1,2,4-triazol-3-one potassium salt, 5-butyl-2,4-dihydro-4-[[2′-[N-(2-methyl-3-furoyl)sulfamoyl]biphenyl-4-yl]methyl]-2-{2-(trifluoromethyl)phenyl]-1,2,4-triazol-3-one, 5-butyl-2,4-dihydro-4-[[2′-[N-(3-methyl-2-thiophenecarbonyl)sulfamoyl]biphenyl-4-yl]methyl]-2-{2-(trifluoromethyl)phenyl]-1,2,4-triazol-3-one, 5-butyl-2,4-dihydro-4-[[2′-[N-(2-thiophenecarbonyl)sulfamoyl]biphenyl-4-yl]methyl]-2-{2-(trifluoromethyl)phenyl]-1,2,4-triazol-3-one, and 5-butyl-2,4-dihydro-4-[[2′-[N-[2-(methypyrrole)carbonyl]sulfamoyl]biphenyl-4-yl]methyl]-2-{2-(trifluoromethyl)phenyl]-1,2,4-triazol-3-one.


SEQUENCE LISTING (19 sequences)

SEQ ID NO: 1 - protein, 9 aa, Artificial
  EP4 receptor antagonist
IFTSYLECL

SEQ ID NO: 2 - protein, 8 aa, Artificial
  EP4 receptor antagonist
IFASYECL

SEQ ID NO: 3 - protein, 8 aa, Artificial
  EP4 receptor antagonist
IFTSAECL

SEQ ID NO: 4 - protein, 8 aa, Artificial
  EP4 receptor antagonist
IFTSYEAL

SEQ ID NO: 5 - protein, 8 aa, Artificial
  EP4 receptor antagonist
ILASYECL

SEQ ID NO: 6 - protein, 8 aa, Artificial
  EP4 receptor antagonist
IFTSTDCL

SEQ ID NO: 7 - protein, 7 aa, Artificial
  EP4  receptor antagonist
XTSYEAL

SEQ ID NO: 8 - protein, 7 aa, Artificial
  EP4 receptor antagonist
XTSYEAL

SEQ ID NO: 9 - DNA, 22 bp, Artificial
  Consensus sequence
caagcaggctaatactgatagg

SEQ ID NO: 10 - DNA, 22 bp, Artificial
  Primer
atctgcctgctctggtcaatgg

SEQ ID NO: 11 - DNA, 22 bp, Artificial
  EP2 Forward Primer
gaccgcttacctgcagctgtac

SEQ ID NO: 12 - DNA, 18 bp, Artificial
  EP2 Reverse Primer
tgaagttgcaggcgagca

SEQ ID NO: 13 - DNA, 27 bp, Artificial
  EP2 Probe
ccaccctgctgctgcttctcattgtct

SEQ ID NO: 14 - DNA, 20 bp, Artificial
  EP4 Forward Primer
acgccgcctactcctacatg

SEQ ID NO: 15 - DNA, 19 bp, Artificial
  EP4 Reverse Primer
agaggacggtggcgagaat

SEQ ID NO: 16 - DNA, 22 bp, Artificial
  EP4 Probe
acgcgggcttcagctccttcct

SEQ ID NO: 17 - DNA, 20 bp, Artificial
  18S Forward Primer
cggctaccacatccaaggaa

SEQ ID NO: 18 - DNA, 18 bp, Artificial
  18S Reverse Primer
gctggaattaccgcggct

SEQ ID NO: 19 - DNA, 22 bp, Artificial
  18S Probe
tgctggcaccagacttgccctc